(19) United States
(12) Patent Application Publication
(10) Pub. No.: US 2022/0251058 A1
(43) Pub. Date: Aug. 11, 2022

(54) METHODS FOR PREPARING CANNABINOIDS BY HETEROGENEOUS-ACID-PROMOTED DOUBLE-BOND MIGRATION

(71) Applicant: Canopy Growth Corporation, Smiths Falls (CA)

(72) Inventors: Christopher ADAIR, Smiths Falls (CA); Ben GEILING, Smiths Falls (CA)

(21) Appl. No.: 17/596,355

(22) PCT Filed: Jun. 11, 2020
(86) PCT No.: PCT/CA2020/050803

§ 371 Date: Dec. 8, 2021

Related U.S. Application Data
(60) Provisional application No. 62/860,155, filed on Jun. 11, 2019.

(51) Int. Cl. C07D 311/80

(52) U.S. Cl. CPC C07D 311/80

(57) ABSTRACT

Disclosed is a method for converting a first cannabinoid into a second cannabinoid that is a regioisomer of the first cannabinoid. The method comprises contacting the first cannabinoid with a Lewis-acidic heterogeneous reagent under reaction conditions comprising: (i) a reaction temperature that is within a target reaction-temperature range for the Lewis-acidic heterogeneous reagent and the first cannabinoid; and (ii) a reaction time that is within a target reaction-time range for the Lewis-acidic heterogeneous reagent, the reaction temperature, and the first cannabinoid.

### CROSS-REFERENCE TO RELATED APPLICATION

This application claims priority to and benefit of U.S. Provisional Patent Application Ser. No. 62/860,155 filed on Jun. 11, 2019, which is hereby incorporated by reference.

### TECHNICAL FIELD

The present disclosure generally relates to methods for isomerizing cannabinoids. In particular, the present disclosure relates to methods for preparing cannabinoids by inducing double-bond migration reactions.

### BACKGROUND

Cannabinoids are often defined in pharmacological terms as a class of compounds that exceed threshold-binding activities for specific receptors found in central-nervous-system and/or peripheral tissues. Such pharmacological definitions are functional in nature, and they encompass a wide range of compounds with, for example: various structural forms (e.g. different fused-ring systems); various functional-group locants (e.g. different arene-substitution patterns); and/or various alkyl-substituent chain lengths (e.g. C₃H₇ vs C₅H₁₁). Accordingly, cannabinoids are also often defined based on chemical structure and, in this context, many cannabinoids are characterized as isomeric cannabinoids. Isomeric cannabinoids are those which share the same atomic composition but different structural or spatial atomic arrangements. For example, Δ¹-cannabidiol (Δ¹-CBD), Δ⁹-tetrahydrocannabinol (Δ⁹-THC) and Δ⁸-tetrahydrocannabinol (Δ⁸-THC) are all isomeric cannabinoids in that they each have an atomic composition of C₂₁H₃₀O₂, but different structural arrangements as shown in SCHEME 1:

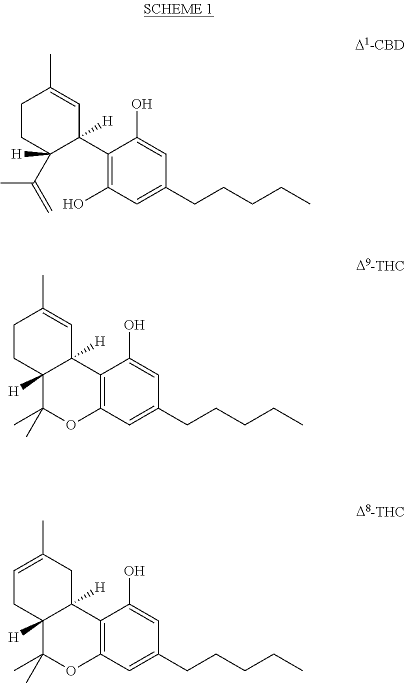 [SMILES: C=C(C)[C@@H]1CCC(C)=C[C@H]1c1c(O)cc(CCCCC)cc1O ; CCCCCc1cc(O)c2c(c1)OC(C)(C)[C@@H]1CCC(C)=C[C@@H]21 ; CCCCCc1cc(O)c2c(c1)OC(C)(C)[C@@H]1CC=C(C)C[C@@H]21]

Compounds that differ only in the location of a particular functional group are known as regioisomers. Hence, cannabinoids that differ only in the location of a particular functional group are known as regioisomeric cannabinoids. Δ⁸-THC and Δ⁹-THC are archetypal regioisomeric cannabinoids—their structures differ only in the location of an alkene functional group. Notably, the cannabinoid-receptor-binding affinity for Δ⁸-THC is similar to that of Δ⁹-THC, but Δ⁸-THC is reported to be approximately 50% less potent in terms of psychoactivity. More generally, small structural changes often correlate with substantial differences in pharmacological properties within cannabinoid classes/subclasses. Moreover, within the various cannabinoid subclasses, regioisomeric cannabinoids often vary greatly with respect to natural abundance. Accordingly, methods for converting cannabinoids into their regioisomeric analogs are desirable.

### SUMMARY

The present disclosure provides methods for preparing cannabinoids by double-bond-migration reactions wherein a first cannabinoid is converted into a second cannabinoid that is a regioisomer of the first cannabinoid. The methods of the present disclosure feature: class III solvents (or are solvent free); mild reaction conditions; scalable protocols; and/or easy-to-separate reagents. Without being bound to any particular theory, the present disclosure posits that these features are engendered by the utilization of acidic heterogeneous reagents having particular acidic properties.

In select embodiments, the present disclosure relates to a method for converting a first cannabinoid into a second cannabinoid that is a regioisomer of the first cannabinoid. In such embodiments, the method may comprise contacting the first cannabinoid with a Lewis-acidic heterogeneous reagent under reaction conditions comprising: (i) a reaction temperature that is within a target reaction-temperature range for the Lewis-acidic heterogeneous reagent and the first cannabinoid; and (ii) a reaction time that is within a target reaction-time range for the Lewis-acidic heterogeneous reagent, the reaction time and the first cannabinoid.

In select embodiments, the present disclosure relates to a method for converting Δ⁹-tetrahydrocannabinol (Δ⁹-THC) into Δ⁸-tetrahydrocannabinol (Δ⁸-THC), the method comprising contacting the Δ⁹-THC with a Lewis-acidic heterogeneous reagent under reaction conditions comprising: (i) a reaction temperature that is greater than about 20° C.; and (ii) a reaction time that is greater than about 1 h.

In select embodiments, the present disclosure relates to a method for converting Δ¹⁰-tetrahydrocannabinol (A¹⁰-THC) into Δ^{10a}-tetrahydrocannabinol (Δ^{10a}-THC), the method comprising contacting the A¹⁰-THC with a Lewis-acidic heterogeneous reagent under reaction conditions comprising: (i) a reaction temperature that is greater than about 20° C.; and (ii) a reaction time that is greater than about 1 h.

Other aspects and features of the present disclosure will become apparent to those ordinarily skilled in the art upon review of the following description of specific embodiments.

### BRIEF DESCRIPTION OF THE DRAWINGS

FIG. 1 shows a high-performance liquid chromatogram for Example 1.

FIG. 2 shows a high-performance liquid chromatogram for Example 2.

### DETAILED DESCRIPTION

As noted above, the present disclosure provides methods for preparing cannabinoids by double-bond migration reactions wherein a first cannabinoid is converted into a second cannabinoid that is a regioisomer of the first cannabinoid. The methods of the present disclosure are suitable for use at industrial scale in that they do not require: (i) complicated and/or dangerous reagent-addition, quenching, and/or work-up steps; and (ii) dangerous, and/or toxic solvents and/or reagents. Importantly, the methods of the present disclosure provide access to one or more cannabinoids that are not naturally abundant in typical cannabis cultivars. For example, the methods of the present disclosure provide access to Δ⁸-THC and Δ^{10a}-THC. Also importantly, the present disclosure provides methods for preparing mixtures of cannabinoid regioisomers in various relative proportions. While pharmacokinetic interactions between mixtures of cannabinoid regioisomers are not well understood, it is expected that access to an array of compositions of wide ranging regioisomeric ratios is desirable in both medicinal and recreational contexts. Moreover, it is expected that access to an array of compositions of varying regioisomeric ratios is desirable to synthetic chemists.

Without being bound to any particular theory, the present disclosure asserts that the double-bond isomerization reactions disclosed herein are associated with the utilization of acidic heterogeneous reagents. The utilization of acidic heterogeneous reagents also appears to be compatible with the use of class III solvents (or neat reaction conditions) which may obviate the need for the dangerous and/or hazardous solvents that are typical of the prior art. The utilization of acidic heterogeneous reagents also allows for product mixtures to be isolated by simple solid/liquid separations (e.g. filtration and/or decantation). As such, the utilization of acidic heterogeneous reagents appears to underlie the cumulative advantages of the present disclosure.

In select embodiments, the present disclosure relates to a method for converting a first cannabinoid into a second cannabinoid that is a regioisomer of the first cannabinoid. In such embodiments, the method may comprise contacting the first cannabinoid with a Lewis-acidic heterogeneous reagent under reaction conditions comprising: (i) a reaction temperature that is within a target reaction-temperature range for the Lewis-acidic heterogeneous reagent and the first cannabinoid; and (ii) a reaction time that is within a target reaction-time range for the Lewis-acidic heterogeneous reagent, the reaction time and the first cannabinoid.

In select embodiments, the present disclosure relates to a method for converting Δ⁹-tetrahydrocannabinol (Δ⁹-THC) into Δ⁸-tetrahydrocannabinol (A⁸-THC), the method comprising contacting the Δ⁹-THC with a Lewis-acidic heterogeneous reagent under reaction conditions comprising: (i) a reaction temperature that is greater than about 20° C.; and (ii) a reaction time that is greater than about 1 h.

In select embodiments, the present disclosure relates to a method for converting Δ¹⁰-tetrahydrocannabinol (A¹⁰-THC) into Δ^{10a}-tetrahydrocannabinol (Δ^{10a}-THC), the method comprising contacting the A¹⁰-THC with a Lewis-acidic heterogeneous reagent under reaction conditions comprising: (i) a reaction temperature that is greater than about 20° C.; and (ii) a reaction time that is greater than about 1 h.

In the context of the present disclosure, the term “contacting” and its derivatives is intended to refer to bringing the first cannabinoid and the Lewis-acidic heterogeneous reagent as disclosed herein into proximity such that a chemical reaction can occur. In some embodiments of the present disclosure, the contacting may be by adding the Lewis-acidic heterogeneous reagent to the CBD. In some embodiments, the contacting may be by combining, mixing, or both. In select embodiments, the first cannabinoid is Δ⁹-THC. In select embodiments, the first cannabinoid is Δ¹⁰-THC.

As used herein, the term “cannabinoid” refers to: (i) a chemical compound belonging to a class of secondary compounds commonly found in plants of genus cannabis, (ii) synthetic cannabinoids and any enantiomers thereof, and/or (iii) one of a class of diverse chemical compounds that may act on cannabinoid receptors such as CB1 and CB2.

In select embodiments of the present disclosure, the cannabinoid is a compound found in a plant, e.g., a plant of genus cannabis, and is sometimes referred to as a phytocannabinoid. One of the most notable cannabinoids of the phytocannabinoids is tetrahydrocannabinol (THC), the primary psychoactive compound in cannabis. Cannabidiol (CBD) is another cannabinoid that is a major constituent of the phytocannabinoids. There are at least 113 different cannabinoids isolated from cannabis, exhibiting varied effects.

In select embodiments of the present disclosure, the cannabinoid is a compound found in a mammal, sometimes called an endocannabinoid.

In select embodiments of the present disclosure, the cannabinoid is made in a laboratory setting, sometimes called a synthetic cannabinoid. In one embodiment, the cannabinoid is derived or obtained from a natural source (e.g. plant) but is subsequently modified or derivatized in one or more different ways in a laboratory setting, sometimes called a semi-synthetic cannabinoid.

In many cases, a cannabinoid can be identified because its chemical name will include the text string “*cannabi*”. However, there are a number of cannabinoids that do not use this nomenclature, such as for example those described herein.

As well, any and all isomeric, enantiomeric, or optically active derivatives are also encompassed. In particular, where appropriate, reference to a particular cannabinoid includes both the “A Form” and the “B Form”. For example, it is known that THCA has two isomers, THCA-A in which the carboxylic acid group is in the 1 position between the hydroxyl group and the carbon chain (A Form) and THCA-B in which the carboxylic acid group is in the 3 position following the carbon chain (B Form). As will be appreciated by those skilled in the art who have benefitted from the teachings of the present disclosure, the terms “first cannabinoid” and/or “second cannabinoid” may refer to: (ii) salts of acid forms, such as Na⁺ or Ca²¹ salts of such acid forms; and/or (iii) ester forms, such as formed by hydroxyl-group esterification to form traditional esters, sulphonate esters, and/or phosphate esters.

Examples of cannabinoids include, but are not limited to, Cannabigerolic Acid (CBGA), Cannabigerolic Acid monomethylether (CBGAM), Cannabigerol (CBG), Cannabigerol monomethylether (CBGM), Cannabigerovarinic Acid (CBGVA), Cannabigerovarin (CBGV), Cannabichromenic Acid (CBCA), Cannabichromene (CBC), Cannabichromevarinic Acid (CBCVA), Cannabichromevarin (CBCV), Cannabidiolic Acid (CBDA), Cannabidiol (CBD), Δ6-Cannabidiol (Δ6-CBD), Cannabidiol monomethylether (CBDM), Cannabidiol-C4 (CBD-C4), Cannabidivarinic Acid (CBDVA), Cannabidivarin (CBDV), Cannabidiorcol (CBD-C1), Tetrahydrocannabinolic acid A (THCA-A), Tetrahydrocannabinolic acid B (THCA-B), Tetrahydrocannabinol (THC or Δ9-THC), Δ8-tetrahydrocannabinol (Δ8-THC), trans-Δ10-tetrahydrocannabinol (trans-Δ10-THC), cis-Δ10-tetrahydrocannabinol (cis-Δ10-THC), Tetrahydrocannabinolic acid C4 (THCA-C4), Tetrahydrocannabinol C4 (THC-C4), Tetrahydrocannabivarinic acid (THCVA), Tetrahydrocannabivarin (THCV), Δ8-Tetrahydrocannabivarin (Δ8-THCV), Δ9-Tetrahydrocannabivarin (Δ9-THCV), Tetrahydrocannabiorcolic acid (THCA-C1), Tetrahydrocannabiorcol (THC-C1), Δ7-cis-iso-tetrahydrocannabivarin, Δ8-tetrahydrocannabinolic acid (Δ8-THCA), Δ9-tetrahydrocannabinolic acid (Δ9-THCA), Cannabicyclolic acid (CBLA), Cannabicyclol (CBL), Cannabicyclovarin (CBLV), Cannabielsoic acid A (CBEA-A), Cannabielsoic acid B (CBEA-B), Cannabielsoin (CBE), Cannabinolic acid (CBNA), Cannabinol (CBN), Cannabinol methylether (CBNM), Cannabinol-C4 (CBN-C4), Cannabivarin (CBV), Cannabino-C2 (CBN-C2), Cannabiorcol (CBN-C1), Cannabinodiol (CBND), Cannabinodivarin (CBDV), Cannabitriol (CBT), 11-hydroxy-Δ9-tetrahydrocannabinol (11-OH-THC), 11 nor 9-carboxy-Δ9-tetrahydrocannabinol, Ethoxy-cannabitriolvarin (CBTVE), 10-Ethoxy-9-hydroxy-Δ6a-tetrahydrocannabinol, Cannabitriolvarin (CBTV), 8,9 Dihydroxy-Δ6a(10a)-tetrahydrocannabinol (8,9-Di-OH-CBT-C5), Dehydrocannabifuran (DCBF), Cannbifuran (CBF), Cannabichromanon (CBCN), Cannabicitran, 10-Oxo-Δ6a(10a)-tetrahydrocannabinol (OTHC), Δ9-cis-tetrahydrocannabinol (cis-THC), Cannabiripsol (CBR), 3,4,5,6-tetrahydro-7-hydroxy-alpha-alpha-2-trimethyl-9-n-propyl-2,6-methano-2H-1-benzoxocin-5-methanol (OH-iso-HHCV), Trihydroxy-delta-9-tetrahydrocannabinol (triOH-THC), Yangonin, Epigallocatechin gallate, Dodeca-2E, 4E, 8Z, 10Z-tetraenoic acid isobutylamide, hexahydrocannibinol, and Dodeca-2E, 4E-dienoic acid isobutylamide.

Within the context of this disclosure, where reference is made to a particular cannabinoid without specifying if it is acidic or neutral, each of the acid and/or decarboxylated forms are contemplated as both single molecules and mixtures.

As used herein, the term “THC” refers to tetrahydrocannabinol. “THC” is used interchangeably herein with “Δ9-THC”.

In select embodiments of the present disclosure, a “first cannabinoid” and/or a “second cannabinoid” may comprise THC (Δ9-THC), Δ8-THC, trans-Δ10-THC, cis-Δ10-THC, THCV, Δ8-THCV, Δ9-THCV, CBD, CBDA, CBDV, CBDVA, CBC, CBCA, CBCV, CBG, CBGV, CBN, CBNV, CBND, CBNDV, CBE, CBEV, CBL, CBLV, CBT, or cannabicitran.

Structural formulae of cannabinoids of the present disclosure may include the following:

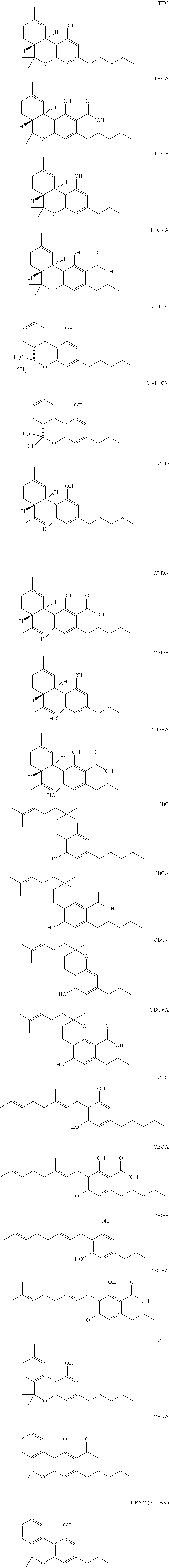

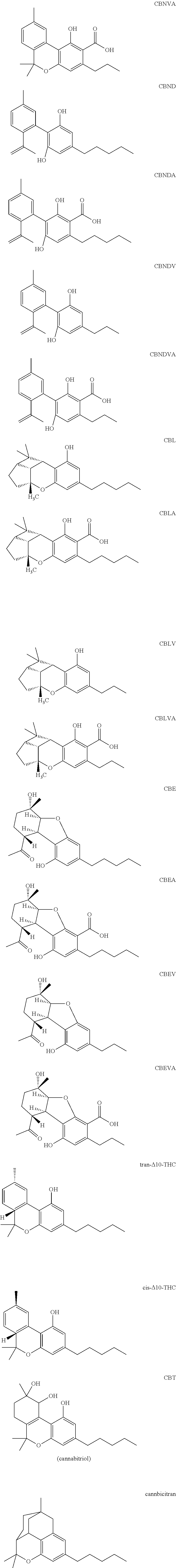

In select embodiments, the first cannabinoid or the second cannabinoid may comprise CBD, CBDV, CBC, CBCV, CBG, CBGV, THC, THCV, or a regioisomer thereof. As used herein, the term “regioisomers” refers to compounds that differ only in the location of a particular functional group.

In select embodiments of the present disclosure, the first cannabinoid is Δ⁹-THC or Δ¹⁰-THC.

In select embodiments, the first cannabinoid is a component of a distillate, an isolate, a concentrate, an extract, or a combination thereof.

In the context of the present disclosure, the relative quantities of a first cannabinoid and a second cannabinoid in a particular composition may be expressed as a ratio—second cannabinoid:first cannabinoid. In select embodiments of the present disclosure, a first cannabinoid may be converted into a mixture of cannabinoid products referred to herein as a second cannabinoid, a third cannabinoid, and so on. The relative quantities of cannabinoid products in a mixture may be referred to with analogous ratios (e.g. second cannabinoid:third cannabinoid). Those skilled in the art will recognize that a variety of analytical methods may be used to determine such ratios, and the protocols required to implement any such method are within the purview of those skilled in the art. By way of non-limiting example, such ratios may be determined by diode-array-detector high pressure liquid chromatography, UV-detector high pressure liquid chromatography, nuclear magnetic resonance spectroscopy, mass spectroscopy, flame-ionization gas chromatography, gas chromatograph-mass spectroscopy, or combinations thereof. In select embodiments of the present disclosure, the compositions provided by the methods of the present disclosure have second cannabinoid:first cannabinoid ratios of greater than 1.0:1.0, meaning the quantity of the second cannabinoid in the composition is greater than the quantity of the first cannabinoid in the composition. For example, the compositions provided by the methods of the present disclosure may have second cannabinoid:first cannabinoid ratios of: (i) greater than about 2.0:1.0; (ii) greater than about 3.0:1.0; (iii) greater than about 5.0:1.0; (iv) greater than about 10.0:1.0; (v) greater than about 15.0:1.0; (vi) greater than about 20.0:1.0; (vii) greater than about 50.0:1.0; and (viii) greater than about 100.0:1.0. In select embodiments of the present disclosure, the compositions provided by the methods of the present disclosure have second cannabinoid:third cannabinoid ratios of greater than 1.0:1.0, meaning the quantity of the second cannabinoid in the composition is greater than the quantity of the third cannabinoid in the composition. For example, the compositions provided by the methods of the present disclosure may have second cannabinoid:third cannabinoid ratios of: (i) greater than about 2.0:1.0; (ii) greater than about 3.0:1.0; (iii) greater than about 5.0:1.0; (iv) greater than about 10.0:1.0; (v) greater than about 15.0:1.0; (vi) greater than about 20.0:1.0; (vii) greater than about 50.0:1.0; and (viii) greater than about 100.0:1.0.

In the context of the present disclosure, a Lewis-acid heterogeneous reagent is one which: (i) comprises one or more sites that are capable of accepting an electron pair from an electron pair donor; and (ii) is substantially not mono-phasic with the reagent. Likewise, in the context of the present disclosure, a Brønsted-acid heterogeneous reagent is one which: (i) comprises one or more sites that are capable of donating a proton to a proton-acceptor; and (ii) is substantially not mono-phasic with the starting material and/or provides an interface where one or more chemical reaction takes place. Importantly, the term “reagent” is used in the present disclosure to encompass both reactant-type reactivity (i.e. wherein the reagent is at least partly consumed as reactant is converted to product) and catalyst-type reactivity (i.e. wherein the reagent is not substantially consumed as reactant is converted to product).

In the context of the present disclosure, the acidity of a Lewis-acid heterogeneous reagent and/or a Brønsted-acid heterogeneous reagent may be characterized by a variety of parameters, non-limiting examples of which are summarized in the following paragraphs.

As will be appreciated by those skilled in the art who have benefitted from the teachings of the present disclosure, determining the acidity of heterogeneous solid acids may be significantly more challenging than measuring the acidity of homogenous acids due to the complex molecular structure of heterogeneous solid acids. The Hammett acidity function (H₀) has been applied over the last 60 years to characterize the acidity of solid acids in non-aqueous solutions. This method utilizes organic indicator bases, known as Hammett indicators, which coordinate to the accessible acidic sites of the solid acid upon protonation. Typically, a color change is observed during titration with an additional organic base (e.g. n-butylamine), which is measured by UV-visible spectroscopy to quantify acidity. Multiple Hammett indicators with pKa values ranging from +6.8 (e.g. neutral red) to −8.2 (e.g. anthraquinone) are tested with a given solid acid to determine the quantity and strength of acidic sites, which is typically expressed in mmol per gram of solid acid for each indicator. Hammett acidity values may not provide a complete characterization of acidity. For example, accurate measurement of acidity may rely on the ability of the Hammett indicator to access the interior acidic sites within the solid acid. Some solid acids may have pore sizes that permit the passage of small molecules but prevent larger molecules from accessing the interior of the acid. H-ZSM-5 may be a representative example, wherein larger Hammett indicators such as anthraquinone may not be able to access interior acidic sites, which may lead to an incomplete measure of its total acidity.

Temperature-Programmed Desorption (TPD) is an alternate technique for characterizing the acidity of heterogeneous solid acids. This technique typically utilizes an organic base with small molecular size (e.g. ammonia, pyridine, n-propylamine), which may react with the acid sites on the exterior and interior of the solid acid in a closed system. After the solid acid is substantially saturated with organic base, the temperature is increased and the change in organic base concentration is monitored gravimetrically, volumetrically, by gas chromatography, or by mass spectrometry. The amount of organic base desorbing from the solid acid above some characteristic temperature may be interpreted as the acid-site concentration. TPD is often considered more representative of total acidity for solid acids compared to the Hammett acidity function, because the selected organic base is small enough to bind to acidic sites on the interior of the solid acid.

In select embodiments of the present disclosure, TPD values are reported with respect to ammonia. Those skilled in the art who have benefited from the teachings of the present disclosure will appreciate that ammonia may have the potential disadvantage of overestimating acidity, because its small molecular size enables access to acidic sites on the interior of the solid acid that are not accessible to typical organic substrates being employed for chemical reactions (i.e. ammonia may fit into pores that a cannabinoid may not). Despite this disadvantage, TPD with ammonia is still considered a useful technique to compare total acidity of heterogeneous solid acids (larger NH₃ absorption values correlate with stronger acidity).

Another commonly used method for characterizing the acidity of heterogeneous solid acids is microcalorimetry. In this technique, the heat of adsorption is measured when acidic sites on the solid acid are neutralized by addition of a base. The measured heat of adsorption is used to characterize the strength of Brønsted-acid sites (the larger the heat of adsorption, the stronger the acidic site, such that more negative values correlate with stronger acidity).

Microcalorimetry may provide the advantage of being a more direct method for the determination of acid strength when compared to TPD. However, the nature of the acidic sites cannot be determined by calorimetry alone, because adsorption may occur at Brønsted sites, Lewis sites, or a combination thereof. Further, experimentally determined heats of adsorption may be inconsistent in the literature for a given heterogeneous acid. For example, ΔH_{0ads} NH₃ values between about 100 kJ/mol and about 200 kJ/mol have been reported for H-ZSM-5. Thus, heats of adsorption determined by microcalorimetry may be best interpreted in combination with other acidity characterization methods such as TPD to properly characterize the acidity of solid heterogeneous acids.

Non-limiting examples of: (i) Hammett acidity values; (ii) TPD values with reference to ammonia; and (iii) microcalorimetry values with reference to ammonia, for a selection of Lewis-acidic heterogeneous reagents in accordance with the present disclosure are set out in TABLE 1.

TABLE 1
Non-limiting examples of: (i) Hammett acidity values;
(ii) TPD values with reference to ammonia; and (iii)
microcalorimetry values with reference to ammonia.

| | | Hammett | TPD | ΔH⁰_{ads} |
|---|---|---|---|---|
| | | Value | NH₃ | NH₃ |
| Acid Reagent | Classification | (H₀) | (mmol/g) | (kJ/mol) |
| Amberlyst-35 | Ion-exchange resin | −5.6 | 5.2 | −117 |
| Amberlyst-15 | Ion-exchange resin | −4.6 | 4.6 | −116 |
| H-ZSM-5 | Microporous | −5.6 < | 1.0 | −145 |
| | aluminosilicate | H₀ < −3.0 | | |
| | (zeolite) | | | |
| H-Beta | Microporous | — | 0.65 | −120 |
| | aluminosilicate | | | |
| | (zeolite) | | | |
| Al-MCM-41 | Mesoporous | — | 0.26 | — |
| | aluminosilicate | | | |
| Montmorillonite | Phyllosilicate (clay) | −1.5 < | 0.18 | — |
| (K30) | | H₀ < +3.2 | | |

In select embodiments of the present disclosure, the Lewis-acidic heterogeneous reagent may have a Hammett-acidity value (H₀) of between about −8.0 and about 0.0. For example, the Lewis-acidic heterogeneous reagent may have a Hammett-acidity value (Hₒ) of between: (i) about −8.0 and about −7.0; (ii) about −7.0 and about −6.0; (iii) about −6.0 and about −5.0; (iv) about −5.0 and about −4.0; (v) about −4.0 and about −3.0; (vi) about −3.0 and about −2.0; (vii) about −2.0 and about −1.0; or (viii) about −1.0 and about 0.

In select embodiments of the present disclosure, the Lewis-acidic heterogeneous reagent may have a temperature-programmed desorption value of between about 7.5 and about 0.0 as determined with reference to ammonia (TPD_{NH3}). For example, the Lewis-acidic heterogeneous reagent may have a temperature-programmed desorption value of between: (i) about 7.5 and about 6.5 as determined with reference to ammonia (TPD_{NH3}); (ii) about 6.5 and about 5.5 as determined with reference to ammonia (TPD_{NH3}); (iii) about 5.5 and about 4.5 as determined with reference to ammonia (TPD_{NH3}); (iv) about 4.5 and about 3.5 as determined with reference to ammonia (TPD_{NH3}); (v) about 3.5 and about 2.5 as determined with reference to ammonia (TPD_{NH3}); (vi) about 2.5 and about 1.5 as determined with reference to ammonia (TPD_{NH3}); (vii) about 1.5 and about 0.5 as determined with reference to ammonia (TPD_{NH3}); or (viii) about 0.5 and about 0.0 as determined with reference to ammonia (TPD_{NH3}).

In select embodiments of the present disclosure, the Lewis-acidic heterogeneous reagent may have a heat of absorption value of between about −165 and about −100 as determined with reference to ammonia (ΔH^{o}_{ads NH3}). For example, the Lewis-acidic heterogeneous reagent may have a heat of absorption value of between: (i) about −165 and about −150 as determined with reference to ammonia (ΔH^{o}_{ads NH3}); (ii) about −150 and about −135 as determined with reference to ammonia (ΔH^{o}_{ads NH3}); (iii) about −135 and about −120 as determined with reference to ammonia (ΔH^{o}_{ads NH3}); (iv) about −120 and about −105 as determined with reference to ammonia (ΔH^{o}_{ads NH3}); or (v) about −105 and about −100 as determined with reference to ammonia (ΔH^{o}_{ads NH3}).

In select embodiments of the present disclosure, the Lewis-acidic heterogeneous reagent may comprise an ion-exchange resin, a microporous silicate such as a zeolite (natural or synthetic), a mesoporous silicate (natural or synthetic) and/or a phyllosilicate (such as montmorillonite).

Lewis-acidic heterogeneous reagents that comprise an ion-exchange resin may comprise acidic functional groups linked to a backbone of the polymer. Lewis-acidic heterogeneous reagents that comprise an ion-exchange resin may comprise, for example, Amberlyst polymeric resins (also commonly referred to as “Amberlite” resins). Amberlyst polymeric resins include but are not limited to Amberlyst-15, 16, 31, 33, 35, 36, 39, 46, 70, CH10, CH28, CH43, M-31, wet forms, dry forms, macroreticular forms, gel forms, H⁺forms, Na⁺forms, or combinations thereof). In select embodiments of the present disclosure, the Lewis-acidic heterogeneous reagent may comprise an Amberlyst resin that has a surface area of between about 20 m²/g and about 80 m²/g. In select embodiments of the present disclosure, the Lewis-acidic heterogeneous reagent may comprise an Amberlyst resin that has an average pore diameter of between about 100 Å and about 500 Å. In select embodiments of the present disclosure, the Lewis-acidic heterogeneous reagent may comprise Amberlyst-15. Amberlyst-15 is a styrene-divinylbenzene-based polymer with sulfonic acid functional groups linked to the polymer backbone. Amberlyst-15 may have the following structural formula:

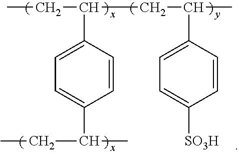

Lewis-acidic heterogeneous reagents that comprise an ion-exchange resin may comprise, for example, Nafion polymeric resins. Nafion polymeric resins may include but are not limited to Nafion-NR50, N115, N117, N324, N424, N1110, SAC-13, powder forms, resin forms, membrane forms, aqueous forms, dispersion forms, composite forms, H⁺forms, Na⁺forms, or combinations thereof.

Lewis-acidic heterogeneous reagents that comprise microporous silicates (e.g. zeolites) may comprise, for example, natural and synthetic zeolites. Lewis-acidic heterogeneous reagents that comprise mesoporous silicates may comprise, for example, Al-MCM-41 and/or MCM-41. Lewis-acidic heterogeneous reagents that comprise phyllosilicates may comprise, for example, montmorillonite. A commonality amongst these materials is that they are all silicates. Silicates may include but are not limited to Al-MCM-41, MCM-41, MCM-48, SBA-15, SBA-16, ZSM-5, ZSM-11, ZSM-22, ZSM-23, ZSM-35, SAPO-11, SAPO-34, SSZ-13, TS-1, KIT-5, KIT-6, FDU-12, Beta, X-type, Y-type, Linde type A, Linde type L, Linde type X, Linde type Y, Faujasite, USY, Mordenite, Ferrierite, Montmorillonite K10, K30, KSF, Clayzic, bentonite, H⁺forms, Na⁺forms, or combinations thereof. Zeolites are commonly used as adsorbents and catalysts (e.g. in fluid catalytic cracking and hydrocracking in the petrochemical industry). Although zeolites are abundant in nature, the zeolites used for commercial and industrial processes are often made synthetically. Their structural framework consists of SiO₄ and AlO₄⁻ tetrahedra, which are combined in specific ratios with an amine or tetraalkylammonium salt “template” to give a zeolite with unique acidity, shape and pore size. The Lewis and/or Brønsted-Lowry acidity of zeolites can typically be modified using two approaches. One approach involves adjusting the Si/Al ratio. Since an AlO₄⁻ moiety is unstable when attached to another AlO₄⁻ unit, it is necessary for them to be separated by at least one SiO₄ unit. The strength of the individual acidic sites may increase as the AlO₄⁻ units are further separated Another approach involves cation exchange. Since zeolites contain charged AlO₄⁻ species, an extra-framework cation such as Na⁺is required to maintain electroneutrality. The extra-framework cations can be replaced with protons to generate the “H-form” zeolite, which has stronger Brønsted acidity than its metal cation counterpart.

In select embodiments of the present disclosure, the Lewis-acidic heterogeneous reagent may comprise “H⁺-form” zeolites “Na⁺-form” zeolites, and/or a suitable mesoporous material. By way of non-limiting example, the acidic heterogeneous reagent may comprise Al-MCM-41, MCM-41, MCM-48, SBA-15, SBA-16, ZSM-5, ZSM-11, ZSM-22, ZSM-23, ZSM-35, SAPO-11, SAPO-34, SSZ-13, TS-1, KIT-5, KIT-6, FDU-12, Beta, X-type, Y-type, Linde type A, Linde type L, Linde type X, Linde type Y, Faujasite, USY, Mordenite, Ferrierite, Montmorillonite, Bentonite, or combinations thereof. Suitable mesoporous materials and zeolites may have a pore diameter ranging from about 0.1 nm to about 100 nm, particle sizes ranging from about 0.1 μm to about 50 μm, Si/Al ratio ranging from 5-1500, and any of the following cations: H⁺, Li⁺, Na⁺, K⁺, NH₄⁺, Rb⁺, Cs⁺, Ag⁺. Furthermore, suitable zeolites may have frameworks that are substituted with or coordinated to other atoms including, for example, titanium, copper, iron, cobalt, manganese, chromium, zinc, tin, zirconium, and gallium.

In select embodiments of the present disclosure, the Lewis-acidic heterogeneous reagent is H-ZSM-5 (P-38 (Si/Al=38), H⁺form, ˜5 angstrom pore size, 2 μm particle size commercially available from ACS Materials), Na-ZSM-5 (P-38 (Si/Al=38), Na⁺form, ˜5 angstrom pore size, 2 μm particle size commercially available from ACS Materials), Al-MCM-41 (aluminum-doped Mobil Composition of Matter No. 41; e.g., P-25 (Si/Al=25), 2.7 nm pore diameter commercially available from ACS Materials), or combinations thereof.

In select embodiments of the present disclosure, a first cannabinoid is contacted with a Lewis-acidic reagent in a protic-solvent system. By way of non-limiting example a protic-solvent system may comprise methanol, ethanol, 1-propanol, 2-propanol, 1-butanol, 2-butanol, water, acetic acid, formic acid, 3-methyl-1-butanol, 2-methyl-1-propanol, 1-pentanol, nitromethane, or a combination thereof.

In select embodiments of the present disclosure, a first cannabinoid is contacted with a Lewis-acidic reagent in an aprotic-solvent system. By way of non-limiting example an aprotic-solvent system may comprise dimethyl sulfoxide, ethyl acetate, dichloromethane, chloroform, toluene, pentane, heptane, hexane, diethyl ether, tert-butyl methyl ether, tetrahydrofuran, dioxane, dimethylformamide, dimethylacetamide, N-methylpyrrolidone, anisole, butyl acetate, cumene, ethyl formate, isobutyl acetate, isopropyl acetate, methyl acetate, methylethylketone, methylisobutylketone, propyl acetate, cyclohexane, para-xylene, meta-xylene, ortho-xylene, 1,2-dichloroethane, or a combination thereof. As will be appreciated by those skilled in the art who have benefitted from the present disclosure, aprotic solvent systems may comprise small amounts of protic species, the quantities of which may be influenced by the extent to which drying and/or degassing procedures are employed.

In select embodiments, the methods of the present disclosure may be conducted in the presence of a class III solvent. Heptane, ethanol, and combinations thereof are non-limiting examples of class III solvents.

In select embodiments of the present disclosure, a first cannabinoid is contacted with a Lewis-acidic reagent under neat reaction conditions. As will be appreciated by those skilled in the art who have benefitted from the present disclosure, neat reaction conditions are substantially free of exogenous solvent.

In select embodiments of the present disclosure, a first cannabinoid is contacted with a Lewis-acidic reagent under reaction conditions characterized by: (i) a reaction temperature that is within a target reaction-temperature range for the particular Lewis-acidic heterogeneous reagent (the particular solvent system where appropriate), and the first cannabinoid; and (ii) a reaction time that is within a target reaction-time range for the particular Lewis-acidic heterogeneous reagent, (the particular solvent system where appropriate), the particular reaction temperature, and the first cannabinoid. As evidenced by the examples of the present disclosure, the acidity of the Lewis-acidic heterogeneous reagent (and the characteristics of the solvent system where appropriate) impact the target reaction-temperature range and the target reaction-time range. Importantly, these reaction parameters appear to be dependent variables in that altering one may impact the others. As such, each reaction temperature may be considered in reference to a target reaction-temperature range for the particular Lewis-acidic heterogeneous reagent, (the particular solvent system where appropriate), the particular reaction time associated with the reaction, and the first cannabinoid. Likewise, each reaction time in the present disclosure may be considered in reference to a target reaction-time range for the particular Lewis-acidic heterogeneous reagent, (the particular solvent system where appropriate) the particular reaction temperature, and the first cannabinoid. With respect to reaction temperatures, by way of non-limiting example, methods of the present disclosure may involve reaction temperatures ranging from about 0° C. to about 200° C. For example, methods of the present disclosure may involve reaction temperatures between: (i) about 5° C. and about 15° C.; (ii) about 15° C. and about 25° C.; (iii) about 25° C. and about 35° C.; (iv) about 35° C. and about 45° C.; (v) about 45° C. and about 55° C.; (vi) about 55° C. and about 65° C.; (vii) about 65° C. and about 75° C.; (viii) about 75° C. and about 85° C.; (ix) about 85° C. and about 95° C.; (x) about 95° C. and about 105° C.; (xi) about 105° C. and about 115° C.; or a combination thereof. Of course, the reaction temperature may be varied over the course of the reaction while still being characterized the one or more of the foregoing reaction temperatures. With respect to reaction times, by way of non-limiting example, methods of the present disclosure may involve reaction temperatures ranging from about 30 minutes to about 85 hours. For example, methods of the present disclosure may involve reaction times between: (i) 30 minutes and about 1 hour; (ii) about 1 hour and about 5 hours; (iii) about 5 hours and about 10 hours; (iv) about 10 hours and 25 hours; (v) about 25 hours and about 40 hours; (vi) about 40 hours and about 55 hours; (vii) about 55 hours and about 70 hours; or (viii) about 70 hours and about 85 hours.

In select embodiments, methods of the present disclosure may involve reactant concentrations ranging from about 0.001 M to about 2 M. For example methods of the present disclosure may involve reactant concentrations of: (i) between about 0.01 M and about 0.1 M; (ii) between about 0.1 M and about 0.5 M; (iii) between about 0.5 M and about 1.0 M; (iv) between about 1.0 M and about 1.5 M; or (v) between about 1.5 M and about 2.0 M.

In select embodiments, methods of the present disclosure may involve Lewis-acidic heterogeneous reagent loadings ranges from about 0.1 molar equivalents to about 100 molar equivalents relative to the reactant. For example methods of the present disclosure may involve Lewis-acidic heterogeneous reagent loadings of: (i) between about 0.1 molar equivalents to about 1.0 molar equivalents, relative to the reactant; (ii) 1.0 molar equivalents to about 5.0 molar equivalents, relative to the reactant; (iii) 5.0 molar equivalents to about 10.0 molar equivalents, relative to the reactant; (iv) 10.0 molar equivalents to about 50.0 molar equivalents, relative to the reactant; or (v) 50.0 molar equivalents to about 100.0 molar equivalents, relative to the reactant.

In select embodiments, the methods of the present disclosure may further comprise a filtering step. By way of non-limiting example the filtering step may employ a fritted Buchner filtering funnel. Suitable filtering apparatus and protocols are within the purview of those skilled in the art.

In select embodiments, the methods of the present disclosure may further comprise a solvent evaporation step, and the solvent evaporation step may be executed under reduced pressure (i.e. in vacuo) for example with a rotary evaporator. Suitable evaporating apparatus and protocols are within the purview of those skilled in the art.

### Exemplary Embodiments

(1) A method for converting a first cannabinoid into a second cannabinoid that is a regioisomer of the first cannabinoid, the method comprising contacting the first cannabinoid with a Lewis-acidic heterogeneous reagent under reaction conditions comprising: (i) a reaction temperature that is within a target reaction-temperature range for the Lewis-acidic heterogeneous reagent and the first cannabinoid; and (ii) a reaction time that is within a target reaction-time range for the Lewis-acidic heterogeneous reagent, the reaction time and the first cannabinoid.

(2) The method of (1), wherein the Lewis-acidic heterogeneous reagent is a Brønsted-acidic heterogeneous reagent.

(3) The method of (1) or (2), wherein the Lewis-acidic heterogeneous reagent has a Hammett-acidity value (Ho) of between about −8.0 and about 0.0.

(4) The method of any one of (1) to (3), wherein the Lewis-acidic heterogeneous reagent has a temperature-programmed desorption value of between about 7.5 and about 0.0 as determined with reference to ammonia (TPD_{NH3}).

(5) The method of any one of (1) to (4), wherein the Lewis-acidic heterogeneous reagent has a heat of absorption value of between about −165 and about −100 as determined with reference to ammonia (ΔHoads NH3).

(6) The method of (1), wherein the Lewis-acidic heterogeneous reagent comprises an ion-exchange resin, a microporous silicate, a mesoporous silicate, a phyllosilicate, or a combination thereof.

(7) The method of (6), wherein the ion-exchange resin is an Amberlyst polymeric resin.

(8) The method of (7), wherein the Amberlyst polymeric resin has a surface area of between about 20 m2/g and about 80 m2/g and an average pore diameter of between about 100 Å and about 500 Å.

(9) The method of (7) or (8), wherein the Amberlyst polymeric resin comprises Amberlyst 15.

(10) The method of (6), wherein the ion-exchange resin is a Nafion polymeric resin.

(11) The method of (10), wherein the Nafion polymeric resin comprises NR50, N115, N117, N324, N424, N1110, SAC-13, or a combination thereof.

(12) The method of (6), wherein the Lewis-acidic heterogeneous reagent is Al MCM-41, MCM-41, MCM-48, SBA-15, SBA-16, ZSM-5, ZSM-11, ZSM-22, ZSM-23, ZSM-35, SAPO-11, SAPO-34, SSZ-13, TS-1, KIT-5, KIT-6, FDU-12, Beta, X-type, Y-type, Linde type A, Linde type L, Linde type X, Linde type Y, Faujasite, Mordenite, Ferrierite, Montmorillonite K10, K30, KSF, Clayzic, bentonite, or a combination thereof.

(13) The method of (12), wherein the Lewis-acidic heterogeneous reagent has a pore diameter of between about 0.1 nm and about 100 nm, a particle size of between about 0.1 μm and about 50 μm, a Si/Al ratio of between about 5 and about 1500, or a combination thereof.

(14) The method of (12) or (13), wherein the Lewis-acidic heterogeneous reagent is H-ZSM-5, with a Si/Al ratio of about 38, a pore size of about 5 Å, and a particle size of about 2 μm.

(15) The method of (12) or (13), wherein the Lewis-acidic heterogeneous reagent is Na-ZSM-5, with a Si/Al ratio of about 38, a pore size of about 5 Å, and a particle size of about 2 μm.

(16) The method of (12) or (13), wherein the Lewis-acidic heterogeneous reagent is Al-MCM-41 with a Si/Al ratio of about 25, and a pore diameter of about 2.7 nm.

(17) The method of any one of (1) to (16), wherein the reaction conditions further comprise a protic-solvent system or an aprotic-solvent system.

(18) The method of (17), wherein the protic-solvent system or the aprotic-solvent system comprises a class III solvent.

(19) The method of (17) or (18), wherein prior to being converted to the composition comprising the second cannabinoid and the third cannabinoid, the first cannabinoid is dissolved in the protic-solvent system or the aprotic-solvent system at a concentration between about 0.001 M and about 2 M.

(20) The method of any one of (1) to (19), wherein the target reaction-temperature range is between about 20° C. and about 100° C.

(21) The method of any one of (1) to (20), wherein the target reaction-time range is between about 10 minutes and about 72 hours.

(22) The method of any one of (1) to (21), wherein the Lewis-acidic heterogeneous reagent has a reagent loading between about 0.1 molar equivalents and about 100 molar equivalents relative to the first cannabinoid.

(23) The method of any one of (1) to (22), further comprising isolating the second cannabinoid from the Lewis-acidic heterogeneous reagent by a solid-liquid separation technique.

(24) The method of (23), wherein the solid-liquid separation technique comprises filtration, decantation, centrifugation, or a combination thereof.

(25) The method of any one of (1) to (24), wherein the first cannabinoid is a component of a distillate, an isolate, a concentrate, an extract, or a combination thereof.

(26) The method of (25), wherein the extract is a crude extract from hemp.

(27) The method of any one of (1) to (26), wherein the first cannabinoid is a cannabidiol, a cannabichromene, a tetrahydrocannabinol, a cannabidivarin, a cannabigerol, a cannabigerovarin, a cannabichromevarin, or a tetrahydrocannabivarin.

(28) A method for converting Δ9-tetrahydrocannabinol (Δ9-THC) into Δ8-tetrahydrocannabinol (Δ8-THC), the method comprising contacting the Δ9-THC with a Lewis-acidic heterogeneous reagent under reaction conditions comprising: (i) a reaction temperature that is greater than about 20° C.; and (ii) a reaction time that is greater than about 1 h.

(29) A method for converting Δ10-tetrahydrocannabinol (Δ10-THC) into Δ10a-tetrahydrocannabinol (Δ10a-THC), the method comprising contacting the Δ10-THC with a Lewis-acidic heterogeneous reagent under reaction conditions comprising: (i) a reaction temperature that is greater than about 20° C.; and (ii) a reaction time that is greater than about 1 h.

(30) The method of (28) or (29), wherein the reaction conditions further comprise a protic-solvent system or an aprotic-solvent system.

(31) The method of (30), wherein the protic-solvent system comprises ethanol.

(32) The method of (30), wherein the aprotic-solvent system comprises heptane.

(33) The method of any one of (28) to (32), wherein the reaction temperature is between about 20° C. and about 100° C.

(34) The method of any one of (18) to (33), wherein the reaction time is between about 1 h and about 36 h.

(35) The method of any one of (18) to (34), wherein the Lewis-acidic heterogeneous reagent is a Brønsted-acidic heterogeneous reagent.

### Examples

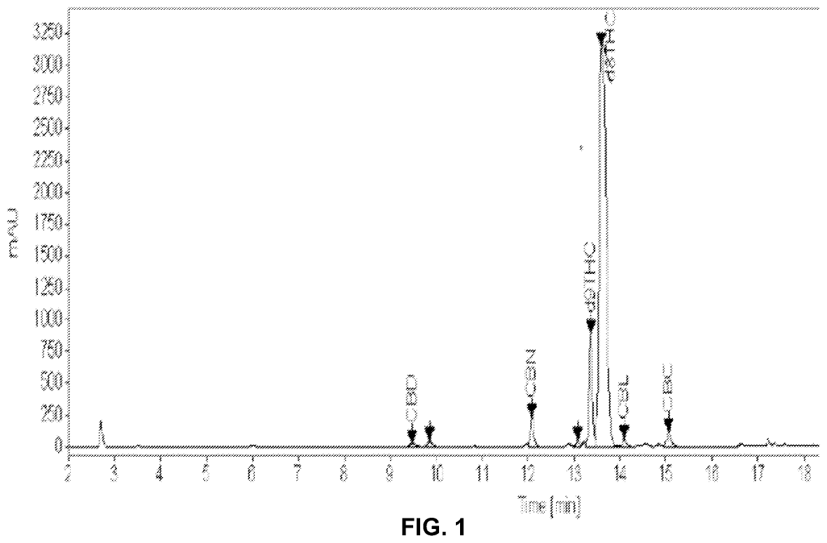

Example 1: To a solution of Δ⁹-THC-rich cannabis extract (500 mg, ˜80% w/w Δ⁹-THC, 0% w/w Δ⁸-THC) in heptane (10 mL) was added Amberlyst-15 (100 mg). The reaction was stirred at room temperature for 18 hours. The reaction was filtered using a fritted Buchner filtering funnel and then the reaction solvent was evaporated in vacuo. Analysis by HPLC (FIG. 1) showed near complete consumption of Δ⁹-THC (11.3% remained) with Δ⁸-THC as the major product (80.2%).

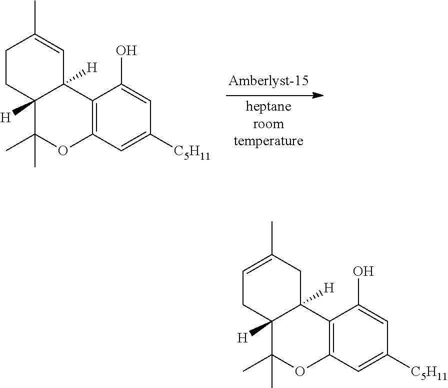

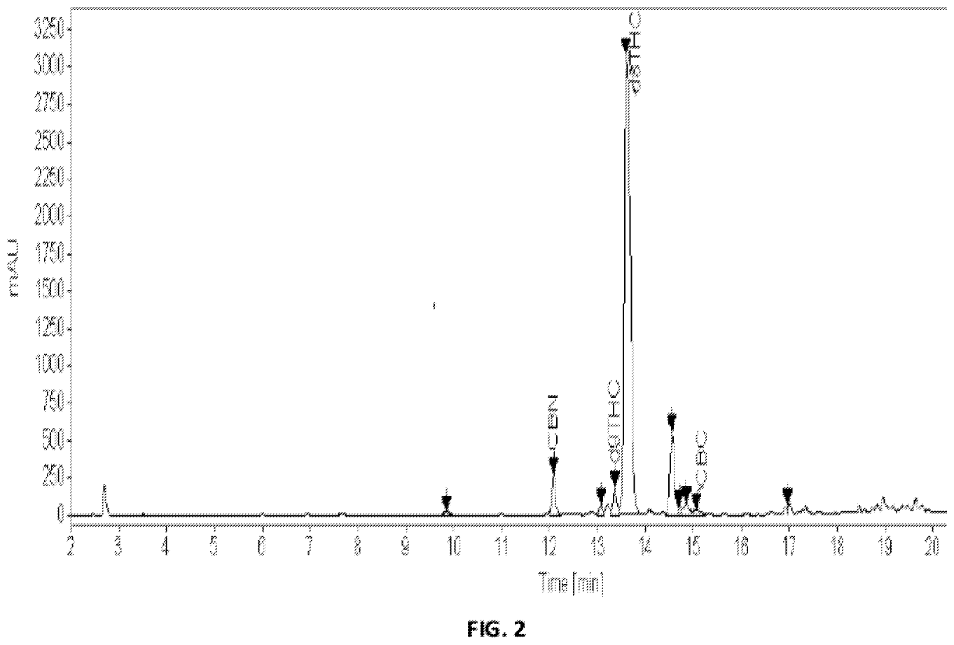

Example 2: To a solution of Δ⁹-THC-rich cannabis extract (500 mg, ˜80% w/w Δ⁹-THC, 0% w/w Δ⁸-THC) in heptane (10 mL) was added Amberlyst-15 (100 mg). The reaction was stirred at reflux for 18 hours. The reaction was filtered using a fritted Buchner filtering funnel and then the reaction solvent was evaporated in vacuo. Analysis by HPLC (FIG. 2) showed near complete consumption of Δ⁹-THC (2.7% remained) with Δ⁸-THC as the major product (75.0%).

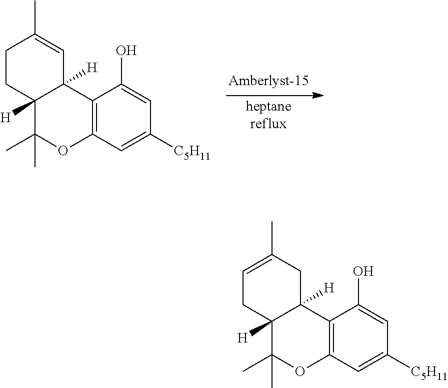

Example 3: To a solution of cis-Δ¹⁰-THC-rich cannabis extract (500 mg) in heptane (10 mL) is added Amberlyst-15 (100 mg). The reaction is stirred at room temperature for 18 hours. The reaction is then filtered using a fritted Buchner filtering funnel and the reaction solvent is then evaporated in vacuo. HPLC is then used to quantify starting material consumption and/or reaction product formation.

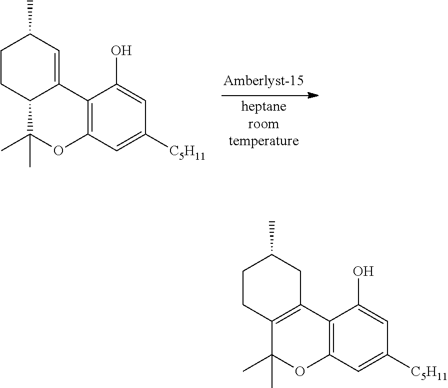

Example 4: To a solution of cis-Δ¹⁰-THC-rich cannabis extract (500 mg) in heptane (10 mL) is added Amberlyst-15 (100 mg). The reaction is stirred at reflux for 18 hours. The reaction is then filtered using a fritted Buchner filtering funnel and the reaction solvent is then evaporated in vacuo. HPLC is then used to quantify starting material consumption and/or reaction product formation.

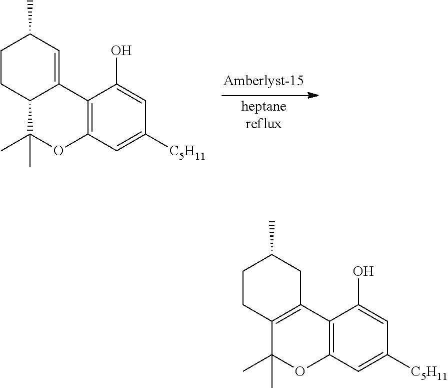

Example 5: To a solution of trans-Δ¹⁰-THC-rich cannabis extract (500 mg) in heptane (10 mL) is added Amberlyst-15 (100 mg). The reaction is stirred at room temperature for 18 hours. The reaction is then filtered using a fritted Buchner filtering funnel and the reaction solvent is then evaporated in vacuo. HPLC is then used to quantify starting material consumption and/or reaction product formation.

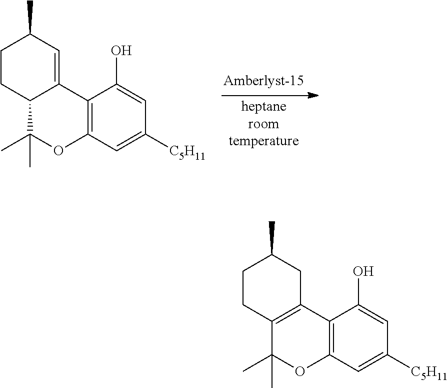

Example 6: To a solution of trans-Δ¹⁰-THC-rich cannabis extract (500 mg) in heptane (10 mL) is added Amberlyst-15 (100 mg). The reaction is stirred at reflux for 18 hours. The reaction is then filtered using a fritted Buchner filtering funnel and the reaction solvent is then evaporated in vacuo. HPLC is then used to quantify starting material consumption and/or reaction product formation.

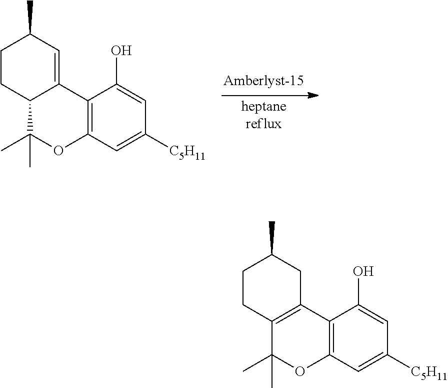

In the present disclosure, all terms referred to in singular form are meant to encompass plural forms of the same. Likewise, all terms referred to in plural form are meant to encompass singular forms of the same. Unless defined otherwise, all technical and scientific terms used herein have the same meaning as commonly understood by one of ordinary skill in the art to which this disclosure pertains.

As used herein, the term “about” refers to an approximately +/−10% variation from a given value. It is to be understood that such a variation is always included in any given value provided herein, whether or not it is specifically referred to.

It should be understood that the compositions and methods are described in terms of “comprising,” “containing,” or “including” various components or steps, the compositions and methods can also “consist essentially of or “consist of the various components and steps. Moreover, the indefinite articles “a” or “an,” as used in the claims, are defined herein to mean one or more than one of the element that it introduces.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, whenever a numerical range with a lower limit and an upper limit is disclosed, any number and any included range falling within the range are specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values even if not explicitly recited. Thus, every point or individual value may serve as its own lower or upper limit combined with any other point or individual value or any other lower or upper limit, to recite a range not explicitly recited.

Therefore, the present disclosure is well adapted to attain the ends and advantages mentioned as well as those that are inherent therein. The particular embodiments disclosed above are illustrative only, as the present disclosure may be modified and practiced in different but equivalent manners apparent to those skilled in the art having the benefit of the teachings herein. Although individual embodiments are dis-cussed, the disclosure covers all combinations of all those embodiments. Furthermore, no limitations are intended to the details of construction or design herein shown, other than as described in the claims below. Also, the terms in the claims have their plain, ordinary meaning unless otherwise explicitly and clearly defined by the patentee. It is therefore evident that the particular illustrative embodiments disclosed above may be altered or modified and all such variations are considered within the scope and spirit of the present disclosure. If there is any conflict in the usages of a word or term in this specification and one or more patent(s) or other documents that may be incorporated herein by reference, the definitions that are consistent with this specification should be adopted.

Many obvious variations of the embodiments set out herein will suggest themselves to those skilled in the art in light of the present disclosure. Such obvious variations are within the full intended scope of the appended claims.

1.-22. (canceled)

23. A method for converting a first cannabinoid into a second cannabinoid that is a regioisomer of the first cannabinoid, the method comprising contacting the first cannabinoid with a Lewis-acidic heterogeneous reagent, optionally in a solvent system, wherein the Lewis-acidic heterogeneous reagent is other than an Amberlyst polymeric resin.

24. The method of claim 23, wherein the Lewis-acidic heterogeneous reagent comprises an ion-exchange resin, a microporous silicate, a mesoporous silicate, a phyllosilicate, or any combination thereof.

25. The method of claim 24, wherein the ion-exchange resin is a Nafion polymeric resin.

26. The method of claim 25, wherein the Nafion polymeric resin is Nafion-NR50, N115, N117, N324, N424, N1110, SAC-13, or a H⁺ or Na⁺form thereof, or any combination thereof.

27. The method of claim 24, wherein the microporous silicate is ZSM-5, ZSM-11, ZSM-22, ZSM-23, ZSM-35, SAPO-11, SAPO-34, SSZ-13, TS-1, Beta, X-type, Y-type, Linde type A, Linde type L, Linde type X, Linde type Y, or any combination thereof.

28. The method of claim 24, wherein the mesoporous silicate is Al-MCM-41, MCM-41, MCM-48, SBA-15, SBA-16, KIT-5, KIT-6, FDU-12, or any combination thereof.

29. The method of claim 24, wherein the phyllosilicate is Faujasite, Mordenite, Ferrierite, Montmorillonite K10, Montmorillonite K20, Montmorillonite K30, Montmorillonite KSF, Clayzic, bentonite, or any combination thereof.

30. The method of claim 23, wherein the solvent system is present.

31. The method of claim 30, wherein the solvent system is an aprotic-solvent system.

32. The method of claim 31, wherein the aprotic-solvent system comprises dimethyl sulfoxide, ethyl acetate, dichloromethane, chloroform, toluene, pentane, heptane, hexane, diethyl ether, tert-butyl methyl ether, tetrahydrofuran, dioxane, dimethylformamide, dimethylacetamide, N-methylpyrrolidone, anisole, butyl acetate, cumene, ethyl formate, isobutyl acetate, isopropyl acetate, methyl acetate, methylethylketone, methylisobutylketone, propyl acetate, cyclohexane, para-xylene, meta-xylene, ortho-xylene, 1,2-dichloroethane, or any combination thereof.

33. The method of claim 30, wherein the solvent system is a protic-solvent system.

34. The method of claim 33, wherein the protic-solvent system comprises methanol, ethanol, 1-propanol, 2-propanol, 1-butanol, 2-butanol, water, acetic acid, formic acid, 3-methyl-1-butanol, 2-methyl-1-propanol, 1-pentanol, nitromethane, or any combination thereof.

35. A method for converting Δ⁹-tetrahydrocannabinol (Δ⁹-THC) into Δ⁸-tetrahydrocannabinol (Δ⁸-THC), the method comprising contacting the Δ⁹-THC with an Amberlyst polymeric resin in an aprotic-solvent system at a reaction temperature that is greater than about 20° C. and below reflux.

36. The method of claim 35, wherein the Amberlyst polymeric resin is Amberlyst-15, 16, 31, 33, 35, 36, 39, 46, 70, CH10, CH28, CH43, M-31, or a H⁺ or Na⁺form thereof, or any combination thereof.

37. The method of claim 36, wherein the Amberlyst polymeric resin is Amberlyst-15.

38. The method of claim 35, wherein the aprotic-solvent system comprises dimethyl sulfoxide, ethyl acetate, dichloromethane, chloroform, toluene, pentane, heptane, hexane, diethyl ether, tert-butyl methyl ether, tetrahydrofuran, dioxane, dimethylformamide, dimethylacetamide, N-methylpyrrolidone, anisole, butyl acetate, cumene, ethyl formate, isobutyl acetate, isopropyl acetate, methyl acetate, methylethylketone, methylisobutylketone, propyl acetate, cyclohexane, para-xylene, meta-xylene, ortho-xylene, 1,2-dichloroethane, or any combination thereof.

39. The method of claim 38, wherein the aprotic-solvent system is heptane.

40. The method of claim 35, wherein the reaction temperature is room temperature.

41. A method for converting Δ10-tetrahydrocannabinol (Δ¹⁰-THC) into Δ^{10a}-tetrahydrocannabinol (Δ^{10a}-THC), the method comprising contacting the Δ¹⁰-THC with an Amberlyst polymeric resin in an aprotic-solvent system.

42. The method of claim 41, wherein the Amberlyst polymeric resin is Amberlyst-15, 16, 31, 33, 35, 36, 39, 46, 70, CH10, CH28, CH43, M-31, or a H⁺ or Na⁺form thereof, or any combination thereof.

43. The method of claim 42, wherein the Amberlyst polymeric resin is Amberlyst-15.

44. The method of claim 41, wherein the aprotic-solvent system comprises dimethyl sulfoxide, ethyl acetate, dichloromethane, chloroform, toluene, pentane, heptane, hexane, diethyl ether, tert-butyl methyl ether, tetrahydrofuran, dioxane, dimethylformamide, dimethylacetamide, N-methylpyrrolidone, anisole, butyl acetate, cumene, ethyl formate, isobutyl acetate, isopropyl acetate, methyl acetate, methylethylketone, methylisobutylketone, propyl acetate, cyclohexane, para-xylene, meta-xylene, ortho-xylene, 1,2-dichloroethane, or any combination thereof.

45. The method of claim 44, wherein the aprotic-solvent system is heptane.
